(19) United States
(12) Patent Application Publication
(10) Pub. No.: US 2011/0124130 A1
(43) Pub. Date: May 26, 2011

(54) DEVICE AND METHOD FOR ANALYSIS OF SAMPLES WITH DEPLETION OF ANALYTE CONTENT

(76) Inventors: Peter Wagner, Menlo Park, CA (US); Frank Zaugg, Redwood City, CA (US); Mark Mitchnik, East Hampton, NY (US)

(21) Appl. No.: 12/936,318

(22) PCT Filed: Apr. 3, 2008
(86) PCT No.: PCT/US08/04372

§ 371 Date: Jan. 27, 2011

(51) Int. Cl. G01N 33/543

(52) U.S. Cl. 436/518; 422/400

(57) ABSTRACT

A system and method for determining the presence and/or concentration of one or more analytes in a sample that comprises a fluid, the system comprising a substrate comprising a sample inlet or inlets and one or more analyte determination flow paths, each analyte determination flow path comprising a defined beginning and a defined terminus and comprising at least one capture zone containing a capture agent for an analyte, the capture agent or agents being immobilized along a portion of the flow path or paths, the flow path or paths being designed so that the one or more analytes are depleted from the sample and bound to the portion of the flow path or paths containing immobilized capture agent or agents, producing an analyte depletion end region for each analyte between the beginning and the terminus of the analyte determination flow path.

### FIELD OF THE INVENTION

This invention relates to systems and methods for determining the presence or concentration of one or more analytes in a sample.

### BACKGROUND OF THE INVENTION

A variety of assay methods and kits suitable for home or lab use are known.

In one common assay format, an analyte-containing sample is applied to a sample-migration medium, such as a test strip or microchannel, and allowed to flow through the medium to a predetermined detection zone, where the analyte is captured by immobilized capture agent. The captured analyte, in turn, may be labeled with a detectable reporter, allowing the presence of analyte in the sample to be determined by the presence or absence of a detectable signal at the detection zone.

It is often desirable, in sample analyte systems, to be able to quantitate the amount of analyte present in the sample, and in other cases, to determine whether a threshold amount of analyte is present in a sample. One limitation of the assay format noted above is the difficulty in quantitating the amount of bound analyte at the detection zone, based solely on the observed level of bound reported in the zone. In general, it is necessary to employ an electronic reader to quantitate or the signal, and this adds to the cost and complexity of the assay system.

It would therefore be desirable to provide a simple flow-through assay device that allows for accurate visual determination of analyte concentration, or threshold level, i.e., without the need for an electronic reader.

### SUMMARY OF THE INVENTION

The invention includes, in one aspect, a method for determining the concentration, or detecting a selected concentration of an analyte in a fluid sample. The method includes the steps of:

(a) introducing the sample into the upstream end of an elongate, analyte-determination flow path containing along its length, an immobilized capture agent effective to bind analyte as the sample migrates through the flow path toward a downstream end of the path,

(b) allowing the sample to migrate through the flow path, wherein analyte in the sample is progressively depleted by binding to immobilized capture agent as it migrates through the path in an upstream-to-downstream direction, producing within the flow path, a region of analyte binding that terminates, at its downstream end, in a depletion end region characterized by progressively less bound analyte on progressing in an upstream-to-downstream direction along the flow path, where the distance of the depletion end region from upstream end of the path is in a specific pre-calibrated relationship to the concentration of analyte in the sample applied to the path,

(c) examining the flow path for the presence of bound analyte, thereby to determine the extent of the analyte binding region along the flow path, and

(d) from the extent of the binding region determined in step (c), determining the concentration or detecting a selected threshold concentration of the analyte in a fluid sample.

For use in determining the concentration of an analyte in a fluid sample, step (c) may include examining the flow path for the presence of bound analyte, thereby to determine the position of the depletion end region along the path, and step (d) may include determining, from the position of the depletion end region determined in step (c), the concentration of the analyte in a fluid sample.

For use in detecting a threshold concentration of an analyte in a fluid sample, step (c) may include examining the flow path for the presence of bound analyte, thereby to determine whether the binding region in the flow path extends beyond a selected path position corresponding to a selected threshold concentration of analyte, and step (d) may include detecting a threshold concentration of analyte in the fluid sample if the binding region in the path extends beyond the selected threshold position.

The capture agent may include binding agents, including antibodies, antibody fragments, and receptors, and nucleic acids, and the analyte is a ligand that forms a specific binding pair with the capture agent.

The examining step may include labeling the analyte with a detectable reporter before or after steps (a) and (b), and detecting the presence of the reporter along the fluid-flow path.

Step (b) may include drawing the sample through the flow pathway by capillarity.

In another aspect, the invention includes a device for determining the concentration, or detecting a selected threshold concentration of an analyte in a fluid sample. The device includes (a) a substrate having formed therein, an elongate analyte-determination flow path containing along its length, an immobilized capture agent effective to bind analyte as a sample, when applied to the upstream end of the path, migrates through the flow path toward a downstream end of the path, wherein analyte in a sample is progressively depleted by binding to immobilized capture agent as it migrates through the flow path in an upstream-to-downstream direction, producing within the flow path, a region of analyte binding that terminates, at its downstream end, in a depletion end region characterized by progressively less bound analyte on progressing in an upstream-to-downstream direction along the flow path, where the distance along the path of the depletion end region from the upstream end of the path is in a specific pre-calibrated relationship to the concentration of analyte in the sample applied to the path, and (b) a readout indicator disposed along a portion of the flow path for indicating:

(i) analyte concentration in the sample as a function of the distance along the path of the depletion end region from the upstream end of the path, where the device is used for determining the concentration of analyte in a sample, and

(ii) a region along the path corresponding to a threshold concentration of analyte, where the device is used for detecting a threshold concentration of analyte in the sample.

For use in determining the concentration of an analyte in a sample, over a selected range of analyte concentrations in a sample, the read-out indicator may extent along a portion of the flow path corresponding to analyte concentrations within the selected range.

For use in detecting a threshold concentration of an analyte in a sample, the read-out indicator may include a window or pointer disposed along the flow path at a position corresponding to the threshold concentration of the analyte.

The device may further includes a labeling reagent for labeling the analyte being tested with a detectable reporter.

The substrate in the device may define a sample-receiving reservoir in fluid communication with the fluid-flow path, and a downstream reservoir for receiving sample fluid exiting from the downstream end of the fluid-flow path.

The flow path in the device may be a channel having a substantially fixed width along its length. The channel may be substantially straight along its length, in one embodiment, or have a serpentine pattern along its length in another embodiment.

The device substrate may have formed thereon, a plurality of such elongate fluid-flow paths, and the immobilized capture agent in each path may be effective to bind one of a plurality of different analytes.

The fluid-flow pathway may be divided into two or more analyte-specific regions, each containing immobilized capture agents effective to bind one of a plurality of different analytes.

The flow path in the device substrate may include structures effective to increase the probability that the analyte, in flowing through the flow path, encounters and binds to a capture agent. The structures may be effective to increase the probability that the analyte, in flowing through the flow path, encounters and binds to a capture agent, are selected from the group consisting of 3-dimensional pillars, increased surface roughness of walls forming the flow path, and beads contained in the flow path.

These and other objects and features of the invention will become more fully apparent when the following detailed description is read in conjunction with the accompanying drawings.

### BRIEF DESCRIPTION OF THE DRAWINGS

FIG. 1A is a plan view of an assay device constructed in accordance with an embodiment of the invention, showing an idealized distribution of bound analyte along the assay device flow path.

FIG. 1B is a graph showing the level of bound analyte on progressing along the length of the device in a typical assay.

FIGS. 2A-2D depict various embodiments of the flow paths that may be used in the devices.

FIGS. 3A-3E depict additional embodiments of the flow paths.

FIG. 4 depicts some optional features of devices according to the invention.

FIG. 5 depicts a series of test results using portions of a device as having a serpentine flow path, showing depletion end regions for several different analyte concentrations.

FIG. 6 shows another portion of a device such as seen in FIG. 5 in which the depletion end region is displayed using a different technique.

### DETAILED DESCRIPTION OF THE INVENTION

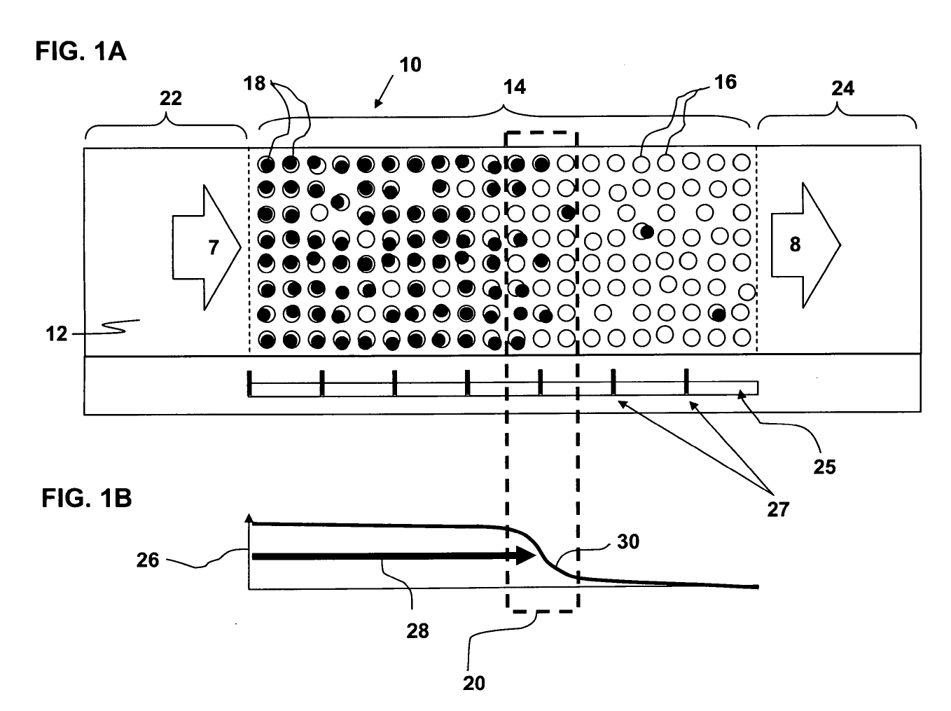

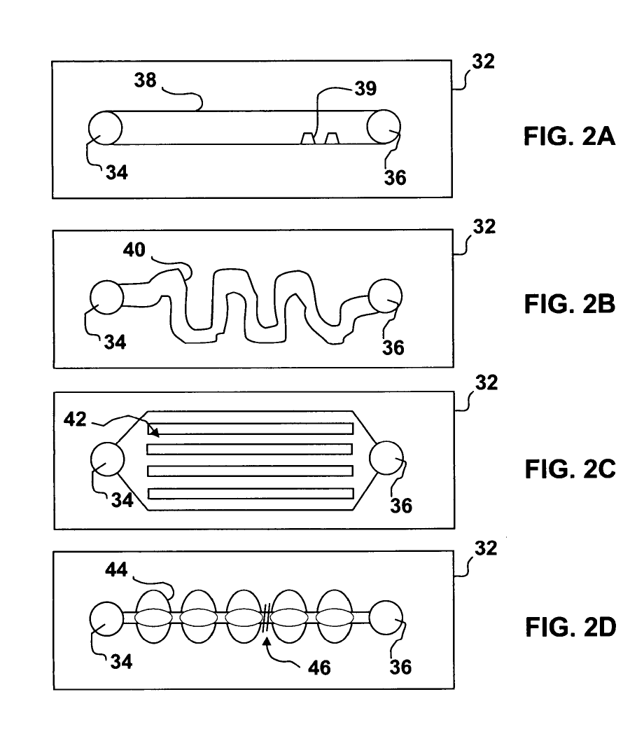

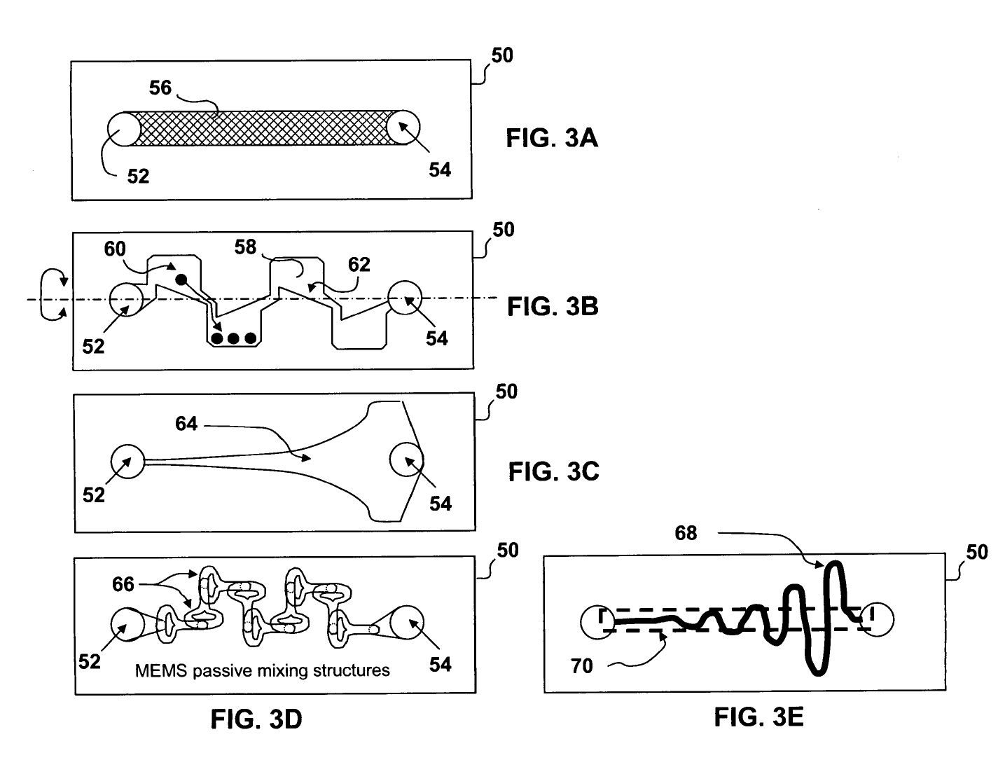

FIG. 1A depicts a sample analyte device 10 according to this invention. As shown in FIG. 1, the device includes a substrate 12 having formed thereon, an elongate analyte-determination flowpath 14 extending along a portion of the length of the substrate. The flowpath, which is shown in idealized view in the figure, has immobilized capture agent, such as indicated by the open circles at 16 in the figure, distributed preferably uniformly along its length. As sample-containing analyte migrates along the flow path, in the upstream-to-downstream direction of arrows 7, 8, the sample analyte is progressively depleted by binding to the immobilized capture agent, as indicated by closed circles at 18, essentially saturating the available binding sites of the capture agent in an upstream-to-downstream direction. At some region along the flowpath, the concentration of analyte in the fluid sample decreases to a point that the density of bound analyte begins to drop off, producing a depletion end region 20 characterized by progressively less bound analyte on progressing in an upstream-to-downstream direction, as shown. It will be appreciated that the relative distance shown in this figure are generally not to scale, in that the flowpath length is generally many times the length of the depletion end region. As will be seen below, e.g., with reference to FIGS. 2 and 3, the device may also include a sample-receiving zone or reservoir for receiving sample upstream of the flowpath, and a downstream reservoir for receiving sample fluid existing from the flowpath.

Also forming part of the device is an indicator scale, such as shown at 25, that is used to determine analyte concentration from the position of the depletion end region along the pathway, or that is used to determine whether a given threshold concentration of analyte is present in the sample, i.e., in a given volume of sample. When used to determine analyte concentration, the indicator scale may include a plurality of analyte-concentration indicia, such as shown at 27, which correlate a given depletion region with a given analyte concentration (amount). That is, the distance of the depletion end region from upstream end of the path is in a specific pre-calibrated relationship to the concentration of analyte in the sample applied to the path.

When used to indicate a threshold level of analyte, the indicator may be a window or single marking as noted below, allowing the user to determine whether the region of uniform analyte binding occurs within the window or at the marking, indicative of a given threshold amount of analyte.

The flow path, also referred to as the “analyte determination flow path” has a defined beginning and a defined terminus or end, where the beginning of the flow path is considered to be the location within the system in which the analyte, after any pretreatment steps, enters a portion of the system that contains capture agent, and the terminus or end of that flow path is considered to be the location at which the sample no longer encounters capture agent. The extent of the flow path that contains immobilized capture agent can vary widely, and can constitute less than half of the flow path length or surface, but preferably, at least a substantial portion of the flow path contains a capture agent or agents. Most preferably a major portion of the flow path will contain capture agent and, in some embodiments, the entire flow path will contain capture agent.

FIG. 1B contains a graphical depiction showing the amount of bound analyte versus the flow path length. The graph indicates at 26 the level of bound analyte as a function of its distance along the flowpath. As seen, over the flowpath region indicated by arrow 28, the analyte is captured at a saturation or near-saturation level, i.e., where the available capture-agent binding sites are largely filled. Within the depletion region 20, there is a drop off in bound analyte, indicated at 30, where the capture agent binding sites are progressively less filled, on progressing in an upstream-to-downstream direction, indicating that the analyte is becoming essentially depleted within the region of migration. As can be appreciated, the total amount of analyte captured, and therefore, the position of the depletion end region, will be determined by the total amount of analyte applied to the device, i.e., the concentration of the analyte in the sample times the total volume of sample applied. For purposes of description herein, the term “concentration” as applied to amount of analyte present, will refer to the level of analyte in a given, known sample volume. That is, for a given sample volume, a higher concentration of analyte means a higher level of total analyte in the sample.

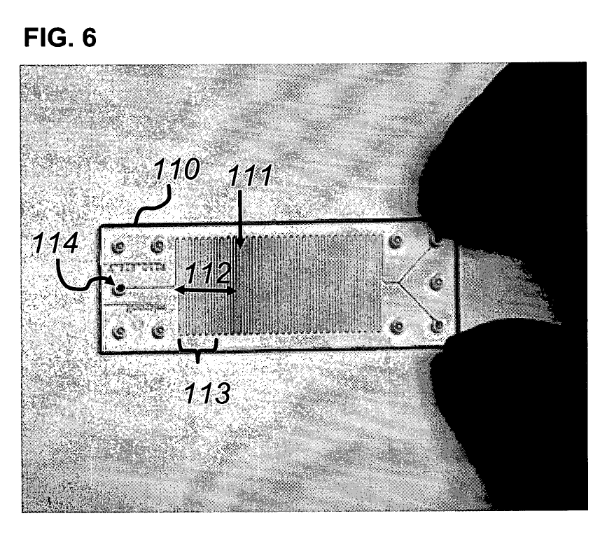

As seen in FIG. 1B, the depletion end region 20 in the graph will exhibit the end of the depleted analyte in a manner that is easily read either by the unaided eye or by an instrument, with or without further staining, depending on the type of analyte tested. Such a result is also depicted in FIG. 6 for an analyte device having a serpentine flowpath. It will be appreciated, e.g., from the sample device shown in FIG. 6, that the flowpath in the device of the invention is generally quite long compared to the expected depletion end region, and typically be at least 5-100 times longer than the expected depletion end region.

The sample can be any liquid, gas or fluid within which one or more specific analytes are to be detected and/or quantified. The analyte may be dissolved or suspended in the fluid, or may be in an emulsion with the fluid. Typical samples include bodily fluids and biopsy or autopsy samples (e.g. blood, blood plasma, blood serum, spinal fluid, joint fluid, eye fluid, feces, urine, saliva, nose-run, tears, sweat, extracted organs, cell slurries or tissue culture supernatants), or fluids extracted or prepared from animals, plants, food, microorganisms or cell cultures. Usable samples also include any liquid, gas or extracted sample obtained in nature (e.g. water samples), or from an industrial or home setting.

The analyte or analytes may be a fluid (liquid or gas), a solid, emulsified, dissolved or suspended material or cellular material. Typical analytes include proteins, antibodies, enzymes, antigens, (poly)peptides, DNA, RNA, lipids, oligonucleotides, cholesterols, sugars, toxins, hormones, messenger molecules, small chemical molecules such as pharmaceuticals and pesticides, as well as macromolecular species such as pollen, whole cells, parts of cells, cell organelles, bacteria, viruses, nanoparticles and pollutants.

The systems, devices and methods of this invention function through depletion of the analyte from the sample onto the surface of the flow path and binding of it to that surface. As seen in FIG. 1B, the level of analyte binding in the depletion end region may be non-linear, meaning that the relationship between the amount of captured analyte, as a function of distance, is non-linear. In the systems and method of this invention initially the analyte typically is captured relatively uniformly along the flow path, but at a location in the flow path (hereinafter referred to as the “analyte depletion end region”) the extent of captured analyte drops off, with a significant drop in the amount of analyte bound to the flow-path surface. The “analyte depletion length”, which may refer either to the length of the flow path that contains captured analyte, or to the overall area of the flow path that contains captured analyte, is either directly or indirectly readable and may then be compared to a calibrated table or ruler (indicator) indicating depletion length versus estimated concentration of the unknown or known analyte in the sample. The use of area (as opposed to the length) of the flow path that contains captured analyte for this determination may occur, for example, when the system contains a non-linear flow path with a reduced flow path-readout window.

The devices of this invention can be built of any suitable material known in the art for making diagnostic or fluidic devices. Preferably, some components of the depletion flow-path are made by injection molding or bonding of polymeric materials (e.g. polystyrene, COC, COP, polycarbonate, or polypropylene). Alternatively, such structures can be created by embossing (polymers) or by various etching/microlithography or micromachining methods (e.g., applied to glass or silicon or other inorganic materials). Suitable structures also can be made by bonding several layers of, e.g., stamped or laser-cut or non-treated thin material foils or by using photopolymer-patterned laminates. Other materials that are known for use in such devices and may be employed in making the devices of this invention are mentioned in, e.g., U.S. Pat. Nos. 6,576,478 and 6,682,942, which are hereby incorporated herein to the extent that their disclosures are not inconsistent with the disclosure herein, and include metals such as gold, platinum, aluminum, copper, titanium and the like, silicon, silica, quartz, glass, and carbon. The devices of this invention can also be composed of other flow path-forming structures, such as tubes, micro channels, or capillaries stretching linearly or bent in a 3-dimensional form, e.g., a capillary tube bent into a spiral. Alternatively, the flow path may consist of a wicking material such as glass fiber or dry-strip material capable of drawing fluid material by capillarity through the strip material.

The flow path for the sample can be a straight path, or it can include curved sections or be composed of curved sections only (e.g., a meandering or serpentine structure). The flow path can also be a vertical channel. The flow path can be a generally open channel or a series of open channels or, alternatively it can be made of a porous material such as nitrocellulose, porous silicon, polymer networks, gel, etc. Again alternatively, the flow path can be composed of a series of chambers that are, or can be placed, in fluid contact with each other. In another embodiment, the flow path can be made of individual flow segments which are separated from each other by structures which can be opened to allow the sample to sequentially move from one segment to the other.

The depletion flow path area can also consist of a plurality of flow path segments arranged in parallel or layered on top of each other, or in another arrangement relative to each other. For determining multiple analytes, the device may contain a plurality of flow paths for the sample. These may be arranged in parallel or in any other convenient manner. For determination of two or more analytes with parallel flow paths, the sample is preferably introduced though a single inlet and removed or collected in a single outlet or downstream chamber, both connected to all of the flow paths. However, a device according to the invention can have multiple injection sites or entry ports, and multiple exit ports or collection chambers, for samples to be analyzed in parallel or for other purposes as described herein. Each of the plurality of flow paths can contain capture agents for different analytes to be determined or the flow paths may serve different purposes. For instance, one flow path may be used to analyze a sample while another may serve for simultaneous calibration. In another embodiment the flow path comprises a series of chambers through which the sample flows, with different chambers containing capture agents for different analytes. For determination of two or more analytes, it is also possible to utilize a single flow path that contains capture agents for different analytes in different portions of the flow path, so that a first analyte can be detected in an upstream segment of the flow path, a second in a middle segment and a third in a downstream segment, for instance. Such an arrangement can be used, though it may require a larger overall device than a device with parallel channels. However, if size of the device is not a significant factor, this embodiment can be quite useful.

The flow path may include structures that improve the mixing of the liquid or enhance or make more frequent the contact of analytes in solution with the capture agent. Embodiments of such structures include passive mixing structures, active mixing elements such as ultrasonic transducers, and MEMS-style mixers.

The flow path can further include structures that increase the surface area containing the capture species. Embodiments of such structures include micro- or mini-pillars, 3-dimensional protruding structures such as macroporous gels, macroporous hard materials such as porous silicon and 3-dimensional nanotube structures composed of various materials, increased surface roughness such as an embossed topography, 3-dimensional polymer networks or structures such as polymer brushes, thin porous layers such as nitrocellulose membranes, sintered spheres of silica or other suitable materials, and bead-loaded flow-path sections.

The flow path preferably is structured such as to maximize the probability that the analyte encounters the capture species in the flow path many times on its travel through the flow path, and also to allow sequential or quasi-sequential depletion of molecular species or other analytes. For example, flow paths in the form of channels are preferably structured such as to provide at least one narrow dimension, and more preferably two (e.g. path width and depth) such that molecules quickly and repeatedly hit the flow-path surface, e.g., by diffusion. Examples of structures having such properties include 3-dimensional open-pore material with pore dimensions in the range of 100 nm to 100 μm, channel-like structures with at least one channel dimension in the range of 500 nm to 500 μm (e.g. channel depth), and multi-pore structures.

For example, one embodiment of the invention contains channels which are 150 μm wide and deep and 600 mm long. Another embodiment contains channels 200 μm wide, 25 μm deep and 1000 mm long. Another embodiment uses a 500 μm-thick nitrocellulose membrane as the flow path.

Some examples of flow-path embodiments that may be used in the devices and methods of this invention are seen in FIGS. 2 and 3.

In FIGS. 2A-2D, for each of the four device shown, 32 indicates the overall general device, 34 indicates a sample inlet, and 36 a sample outlet or means for collecting spent sample. In the FIG. 2A embodiment, the flow path 38 is a straight channel, designed as described above, and coated with a capture agent for an analyte. Preferably single-channel devices of this type are used to analyze for a single analyte, although, as described above, they may be used to determine two or more analytes. The channel optionally contains three-dimensional structures, represented by pillars 39, to increase the channel surface area, improve fluid mixing, reduce the flow rate or reduce the effective pore size. These structures may extend along the entire length of the flow path, may extend into the flow path from opposite surface of the flow path, and may themselves be coated with immobilized capture reagent.

Another type of flow path, shown in FIG. 2B, is a meandering or serpentine channel 40. This type of flow path enables the device to include a relatively long sample flow path in a relatively small device.

In FIG. 2C, 42 depicts parallel multiple flow paths, which may be open or closed channels or porous material, connected to a common sample inlet and common outlet or collection means. These flow paths optionally contain the types of structures mentioned above to enhance contact, mixing and the like. This embodiment of the invention may be used for analysis of a plurality of analytes, by having each channel contain a capture agent for a different analyte. Alternatively one or more of the parallel channels may be used for calibration and/or for references and/or controls.

In FIG. 2D, 44 depicts a series of interconnected chambers that form the flow path. Optionally the flow path contains one or more active or passive valves 46 between chambers that can be opened at specific moments during the assay, and serve for example, the purpose of preventing backflow of sample or to provide longer residence times leading to improved depletion capture of the analyte to the capture surface. Again, as described above, such an embodiment can be used to determine a single analyte or a plurality of analytes.

In FIGS. 3A-3E, for each of the four device shown, 50 indicates the overall general device, 52 indicates a sample inlet, and 54, a sample outlet or means for collecting spent sample. In FIG. 3A, flow path 56 is defined or filled with a porous material, as described above. In FIG. 3B, flow path 58 comprises a series of chambers, such as chambers 60, 62, that are not in a straight trajectory. This embodiment is particularly useful for analyzing a sample that contains an analyte that tends to sediment under gravity, such as cells. Here the chambers are connected by inclined passageways so that the device can be rotated or turned over to propel the analyte from chamber to chamber with minimal blockage or sample backflow. The passages connecting the chambers may contain capture agents.

Flow path 64 in FIG. 3C has a non-constant channel cross-section, for instance to increase the dynamic range of the device. The same can be achieved e.g. by a non-linear bending flow path as depicted in 68 in FIG. 3E, and providing a reduced flow path-readout window as schematically shown at 70 in FIG. 3E. FIG. 3D show a flow path 66 composed of a series of overlapping flow chambers.

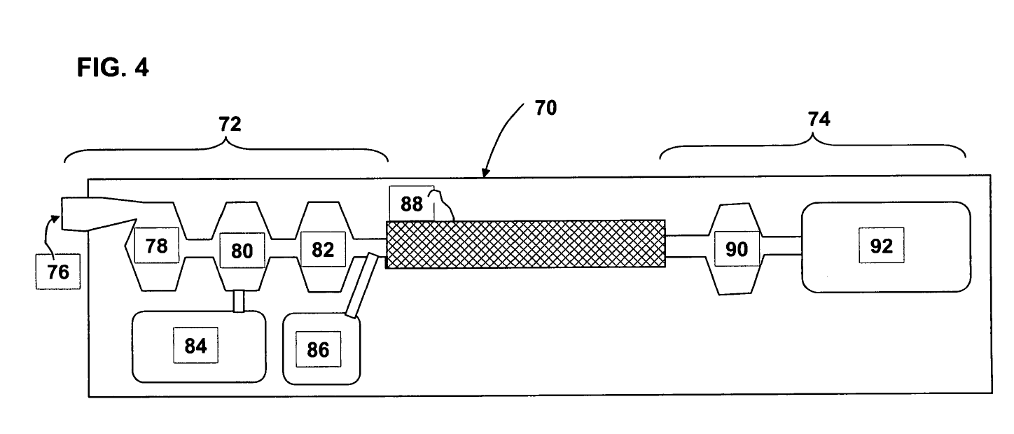

FIG. 4 depicts some optional features in an assay device 70 that may be present in the zones upstream and downstream of the flow path. The upstream sample processing zone, indicated generally at 72, will include some means for introducing a sample into the device. This can include a sampling device, e.g., a finger prick needle to sample blood, a sample injection septum port, or a sample injection cavity. Another optional upstream feature is a structure 76 used to meter or dose a specific sample volume to be passed through the depletion flow path. Such a feature could include a defined volume injection structure similar to a syringe or pipette or a microfluidic overflow sampling compartment allowing excess liquid to go into, e.g., an overflow compartment. Other possible upstream sample processing structures may include areas designed for sample pre-treatment, diluting, concentrating, pre-fractioning or filtration (78), areas designed to remove undesired molecular or cellular species in the sample that could interfere with the device principle (e.g., a pre-chamber with immobilized capture agents to specifically capture interfering substance(s)), or a capture layer located behind a dialysis membrane to selectively only capture or remove molecular species of a defined size.

The devices according to this invention can further comprise other reagent or fluid compartments that contain reagents or solvents required for carrying out the assay. These compartments may be in liquid contact with the depletion flow path or may be controlled by passive or active valves. Such optional upstream features shown in FIG. 4 include a sample labeling zone (80), a reagent reservoir (84), and a secondary reagent or pre-wetting fluid reservoir (86). Devices according to the invention can include any or all of the optional features shown in FIG. 4, or may include none of them. Other optional items that may be included in the devices of the invention include barcodes or other identifying labels, company logo, expiration date, a shelf life/storage conditions label, and sensors that indicate whether devices have been exposed to certain environmental conditions (e.g. elevated temperature or humidity conditions, etc).

Preferably, the quantity of the sample introduced into the device is kept to a certain volume for best results. This can be achieved by means such as streaming the sample through the device for a specific time at a specific flow rate, designing a limited and reproducible suction capacity into the device (using e.g. a defined size of a capillary-action suction pad), initially injecting a defined sample volume into the test strip, or active metering of a defined liquid volume via valves, pumps, or flow regulators, and associated electronics.

FIG. 4 also shows features that typically will be contained in the device downstream of the flow path, in a region indicated generally at 74. As shown, this region may contain one or both of a positive or negative control area (90) that indicates that the device is working satisfactorily. Typically a positive control area will contain an indicator that the sample has flowed through the device, for instance a substance that changes color or becomes colored when contacted with the analyte carrier fluid. A negative control area will indicate that the sample has not flowed properly through the device. The device may also contain a waste reservoir (92) to prevent physical contact of the user with the sample and allow safe disposal. Alternatively, instead of the reservoir the device may contain an exit port through which depleted sample can be removed from the device. The device may also contain a sucking pad to propel the sample through the flow path or paths.

The propulsion of the sample through the flow path or paths can be achieved via various methods. These include passive propulsion, gravity-based movement of the fluid in the desired direction, capillary action provided by appropriate flow-path dimensions with appropriate wetting properties, or by having the sample flow driven by a capillary action material such as an absorptive wick (e.g. filter paper or advanced suction materials or coatings). The wick can either be positioned at the end of the flow-path or the flow path can itself be constituted of a wicking material or other structures that create a capillary action within the flow-path. For depletion of macromolecular or particulate species, the flow path(s) can also be structured such as to, e.g., use gravity to propel the sample. Flow paths of this type can be constituted of several chamber-like structures which are contacted with each other by liquid bridges. Gravity is used to move the particles from one compartment to the other, sequentially. See, e.g. FIGS. 3B and 3D. Alternatively, an evaporative pad can be used to pull liquid through the device by the controlled evaporation of liquid in a wet pad at one extremity of the flow path.

Active propulsion of the sample through the device may be achieved by use of a pumping mechanism which may be external or internal (integrated), e.g., an external pump and/or a MEMS-style pump, via centrifugation such that the liquid is propelled in the desired direction, by applying a negative pressure at the end of the device (e.g., using a syringe, evaporation patch or vacuum or capillary suction-pad), by pressing the liquid forward through the device by a positive pressure applied by, e.g., a syringe or syringe-like device, or by pressing an enclosed compressible liquid compartment with the force of, e.g., the fingers, or by electro-osmotic or electro-kinetic flow.

The capture agent can be any molecule or matrix which can selectively bind one or several analytes. Preferably the capture agent has a high affinity and specificity for the molecular species to be detected and/or quantified, with little or no cross-reactivity to other species.

In a preferred embodiment, the capture agent is a protein, notably an antibody or a fragment thereof, a receptor, an enzyme, or a protease. In another embodiment, the capture agent is an oligonucleotide or polynucleotide, aptamer, an artificially generated protein-binding scaffold, or a phage. In another embodiment, the capture agent is a peptide, oligo- or polysaccharide, or phospholipid. In another embodiment, the capture agent is a small molecule, a drug, a non-biological polymer or a supramolecular structure. If the analyte is known to have an affinity to another species, that other species can potentially be used as the capture agent. The depletion flow path may also be coated with several different capture species which are specific for the same or different analytes. This expedient can be used to increase the binding strength to the analyte, to probe for different epitopes of an analyte, or to measure several different analyte species within the same flow path.

In the systems or devices of the invention, the capture agents are adhered or bound to a solid substrate. The substrate may consist of a material of construction of the device, as described above, and may include a coating or gel. Adherence or placing of the capture agent on the depletion flow path can be achieved through various methods as known in the art, for example by binding the capture species to the substrate using methods such as those described in U.S. Pat. Nos. 6,329,209, 6,365,418, 6,576,478, 6,406,821, 6,475,808, 6,630,358, and 6,682,942, which are hereby incorporated herein to the extent that their disclosures are not inconsistent with the disclosure herein.

The capture agent can be specifically or non-specifically immobilized on the surface of the depletion flow-path. It can be integrated into the material of the flow path itself, it can be formed at the surface of the flow path or it can be indirectly attached to the surface of the flow path by one or several interface layers. Examples of such interface layers include organosilanes, alkanethiol-based or disulfide-based self-assembled monolayers, copolymers, inorganic layers, bifunctional crosslinkers, hydrogels or passively adsorbed proteins such as avidin or albumin species.

The flow-path surface can further be modified with a plurality of different molecular species, e.g., by using certain moieties to promote the binding of the analytes and others to prevent the non-specific adsorption of other components that may be present in the sample. This approach can also be used to dilute the density of capture agents on the surface, e.g., to adjust the dynamic range in which the assay is operating.

The capture agent density, or the relative abundance of capture agent, can be deposited along the length of the flow path in a linear or nonlinear gradient. The capture agent density could be in an exponential, increasing gradient along the depletion path length. This method can be used to extend the dynamic sensitivity range of the test device. The capture agent can also be deposited in sequential or parallel patches of varying density.

Alternatively, the capture agents can be deposited in the flow-path in discrete areas, using e.g. a micro-arraying tool, ink jet printer, spray, pin-based contact printing or screen-printing method. The regions between discrete capture agent areas can be modified with non-binding molecular species or blocked with methods known in the art (e.g. using BSA solutions in the case of protein depletion assays, etc.).

The capture agents can be further deposited in nano-, micro- and macro-patterns, allowing for e.g. diffractometric readout or by other optical interference mechanisms. The capture agents can further be deposited in such patterns as to prevent clogging or crowding of the flow path by immobilized analyte.

It may be necessary to keep the device, or at least that portion of it containing the capture agent, dry, moist, lyophilized or otherwise preserved in order to maintain its activity during storage. Possible means for such preservation include lyophilization of the capture agents or the use of preservative solutions (e.g., protein- or sugar-based solutions) first applied, and then dried, onto the capture layer. Alternatively the device can also be kept or stored fully pre-loaded with a storage, preservation or pre-wetting fluid.

Analyte capturing may also be done by mixing or exposing the analyte capture agent to the analyte before the sample is run through the flow path. In this method the capture agent has a secondary tag or epitope which can then be captured by a second capture agent in the flow path while the analyte is bound to its capture agent. One possible embodiment of this approach is the use of analyte-specific antibodies linked to biotin, with the depletion flow path coated with avidin species to capture the biotinylated antibodies. The non analyte-bound capture agent can be removed from the sample, e.g., through a size-excluding material in a pre-section to the depletion flow path or by selectively binding that capture fraction to a species behind a size-selective membrane (e.g., a dialysis membrane of selected pore size). It is also possible to use a cascade of capture agents (e.g., the device can contain a sandwich immunoassay with multiple interaction partners).

In order to visualize the depletion length or the sections of the flow path which contain bound analyte molecules (or do not contain, for example, if the assay is a competitive assay), different labeling or detection methods can be used. In one embodiment, the device employs a label-free method in which the presence or absence of captured molecules is visible without a label. Such detection can be accomplished if the analytes are large, e.g., cells or other particles, or if the analyte is stained or intrinsically colored such that it can be detected without additional label or stain. A magnifying device such as a lens or microscope may be needed to carry out the readout.

In one preferred embodiment labeled detection antibodies are used. They are allowed to bind to the analyte either before or after the sample is flushed over the depletion flow path. The labeled antibodies specifically bind to the analyte and make it detectable by the unaided eye, colorimetrically or by other optical methods such as fluorescent or colorimetric readers, depending on the type of label used. The labels on the detection species can be any moiety typically used in the art for such purposes, including fluorescent dyes, colored beads or microspheres, gold or silver or other nanoparticles, radioactive species, quantum-dots, radio-tags, Raman tags, chemiluminescent labels, organic stains, etc.

Alternatively, any enzyme-amplified detection mode can be used, as is typically implemented for the readout of microtiter plate-based assays. Possible embodiments of such detection species are antibodies linked to, e.g., peroxidases, phosphatases or dehydrogenases, which are used in combination with an appropriate colorimetric enzyme substrate. For instance, an HRP-linked detection antibody can be used in combination with TMB as the enzyme substrate, leading to a blue substrate product in those depletion flow path areas which contain the captured analyte.

In a preferred embodiment, the areas containing analytes with bound detection species become visible to the unaided eye and can easily be distinguished from the areas with significantly less, or no, bound analyte. For cells, for instance, non-specific or specific cytoplasmic labeling, non-specific or specific cell membrane labeling with fluorophores of colored beads, or non-specific or specific nuclear labeling with fluorophores of colored beads, can be used. Cell labeling can be done in a separate reaction compartment or channel, or together with other processes in a reaction compartment or channel.

The devices of the invention may include elements that enhance the ability to read out the depletion length (e.g. readout contrast). Such elements include materials of different optical clarity and reflectivity, polarizing elements, micro-lens arrays, micro-lenses, LED lights, etc. Several different detection species may be run in parallel through a flow path or through parallel flow paths to detect various analytes in parallel. The detection species may have to exhibit different colors or optical properties so as to allow the unaided eye or the detection unit to differentiate between the different detection species.

The method can be used to determine the presence or absence of a specific analyte (non-quantitatively) in a sample, or to quantify it relative to an internal, external or factory-calibrated standard.

The binding of the analyte to the capture agent may be covalent, ionic, electrostatic or through any other type of interaction. The binding may be reversible or irreversible and may necessitate that the readout is done within a predefined time interval after starting or ending the depletion assay run.

Certain embodiments of the invention may use electronic and/or optical read-out devices to perform the quantification of the assay readout. Such devices can include hand-held devices connected to microprocessors, specifically designed analytical instrumentation and readout devices which can transmit the readout information wirelessly to data receiving/distribution centers.

The depletion length or area readout can be done by any method known in the art. These include reading the depletion length or area using electrochemical methods, by measuring the change in electrical conductivity (along the depletion flow path or orthogonal thereto), or by detecting a change in optical parameters (e.g., using a photosensor array positioned in close proximity to the flow path). Other test-result readout modes may include diffractometric methods in which the capture molecules are arranged in defined patterns on the flow-path surface, forming a diffraction grating which can be read by a laser, and methods based on using liquid crystal technology to visualize the depletion length (e.g. linking the detection species to optically active molecules which change the polarization of light and can thus be read via liquid crystal display technology). However, especially for use in resource-poor areas, a preferred embodiment of the invention allows the readout by the unaided eye, without the need for any electronic or external detection instrumentation.

The readout may be done relative to a lateral reference ruler or a colored or gray-scale structure reference printed or included on or in the depletion flow path. Alternatively a reference scale may be separately provided with the test device.

The devices of the invention may include positive or negative control areas or zones which may be included in parallel to, before, after or within the depletion flow path. Such control zones may, e.g., be used to verify that the sample liquid completely flows through the depletion flow-path, or that certain assay reagents are still active when the assay is carried out, or that the calibration of the device is still accurate. Embodiments of such control areas may include areas coated with reagents that change in color when wetted, or areas containing immobilized antibodies specific to molecules in the sample, or to the detection species, or to reagents contained in the assay kit. The device may further incorporate a reference sample which can be run in a separate depletion flow path of the device.

Access to the test results can be accomplished by several means. In one embodiment the flow path is exposed to the atmosphere and can be read directly. In another embodiment a transparent cover is placed over the flow path for protection against contamination. Again, the readout can be taken directly by the unaided eye or by an instrument. In another embodiment the flow path is covered, but a transparent “window” is provided over that area of the flow path that would show a labeled depletion end region at a certain concentration. Such a device could be used for readily available “yes/no” determination of whether a given analyte is present in a sample at a certain concentration, for instance the legal maximum or minimum concentration for a particular drug. If the analyte is present in the sample at that concentration the label will be detectable through the window; otherwise it would not be detected.

The assay time typically is in the range of from about 30 seconds to about 30 minutes. In some embodiments the assay may take only a few seconds to a few minutes to run. In other embodiments, however, the assay may take several hours or even days. The assay may run on its own once the sample has been introduced, or one or more user intervention steps may be required during the assay. The assay may also include features which direct the user to perform certain tasks after receiving specific signals from the device. Such tasks may e.g. include pressing certain assay cartridge features, e.g., to inject an enzyme substrate into the depletion flow path after running an assay detected by an enzyme amplified detection mode.

Good shelf life stability can be achieved by implementing a liquid reagent-less test strip design. In such a device chemicals or biochemicals may be immobilized on surfaces and then preserved by preserving agents such as trehalose. After preservation, the strips are dried and then sealed into a pouch with or without a drying agent (desiccant pouch) and/or an inert gas filling.

A typical sequence of events in running the depletion assays of the invention would be as follows:

1) Insertion of the sample
2) Optionally, sampling of a defined sample volume by an upstream cartridge feature
3) Optionally sample pre-treatment, e.g. to remove unwanted species from the sample
4) Optionally labeling of the analyte in solution by soluble labeled antibodies
5) Flowing the sample through the depletion flow path.
6) Optional labeling of the analyte in solution by soluble labeled antibodies
7) Reading the depletion length of the analyte in the depletion flow path.
8) Comparing the depletion length to an integrated calibration standard to determine the initial concentration of the analyte in the sample.
9) Discarding the device.

According to one embodiment of the invention, the assay device is designed for use in identifying and monitoring the immune status of HIV-positive patients, by determining CD4 cell count in a subject's blood sample. In this approach, whole blood samples are funneled through a channel architecture integrated into a test strip having walls coated with one or more specific anti-CD4 capture agents. As the blood flows through the channel, CD4 cells adhere to the channel walls, thereby depleting the blood sample from CD4 cells not crossing a pre-calibrated boundary with an analyte depletion end region being detectable at a pre-calibrated location if the cell count is below a certain level.

A device suitable for the cell-assay method may integrate all the necessary sample pre-treatment reaction steps and will allow visual determination of the T-cell count directly from the test strips. Because of the extreme shelf-life conditions that would be encountered in tropical or arid areas, liquid-based protein solutions should be avoided in devices for use under such conditions. Thus, such devices utilize dried, but preserved (protein) reagents on the strips. Such dry reagents can be engineered to have excellent storage stability and assay performance. The strips will also integrate a positive control for verifying the correct functioning of the T-cell test. A waste reservoir which will allow hermetically sealing of the device will allow the disposal of the devices after use without the risk of infecting personnel from blood samples.

In the cell-assay method a defined amount of blood drawn from a finger-prick is either injected into the strip through a port, or, alternatively, a finger-pricking element can be integrated directly into the plastic device. The blood sample is then pushed through the different reagent chambers e.g. by centrifugation (e.g., a small hand-driven centrifuge) or via other mechanical mechanisms known in the art. After sampling, a defined blood volume (constant volume mechanism), is transported into a first reaction chamber to remove any potentially interfering non-T-cell species from the sample solution (e.g. by anti-CD14 capture antibodies immobilized onto the walls). The blood sample is then transferred into an optional second reaction chamber, in which the T-cells can be labeled for easier visual detection further downstream (e.g., by cytoplasmic staining). The labeled T-cells then reach a long depletion channel coated with anti-CD4 capture agents. By careful optimization of the binding capacity, microfluidic properties and surface area in that channel, the CD4⁺T-cells will quantitatively deplete from solution by binding to the flow path surface. A reference guide is provided to ascertain concentration of the cells in the sample. The length of the depletion channel that is visibly coated with labeled CD4⁺ T-cells will be in a pre-calibrated relation to the T-cell count. Further downstream, a small window with, e.g., antibodies against the cell dye, will allow verification that the test-strip is still functional (positive control). Ultimately, the used blood sample reaches a waste reservoir at the end of the test strip.

A semi-quantitative readout based on defined cut-offs for the CD4⁺ T-cell count can be achieved by directly integrating a visual readout into the test strip, without the need for a separate reader. The cell quantification approach in this method is based on sequentially depleting all the CD4⁺ cells present in a defined blood volume onto the walls or surfaces of a micro channel or of material contained in it, and then determining the length of the channel that is coated with cells as a direct measure of the cell count. Compared to methods based on quantifying the intensity of e.g. labels previously attached to T-cells, this method is independent from the labeling efficiency, requires no separation steps and can be done using surface-attached capture molecules (no liquid reagents nor separation/lysis of the erythrocytes are needed).

Through careful adjustment, the design of the flow path in the above cell-assay device will allow the formation of a very sharp boundary between areas with and without cells attached to the walls of the microchannel. The depletion border or end region is clear and, using the guide, indicates the concentration of CD4⁺ cells in the sample. The positive control shows that the device functioned properly, and waste sample has been collected in reservoir.

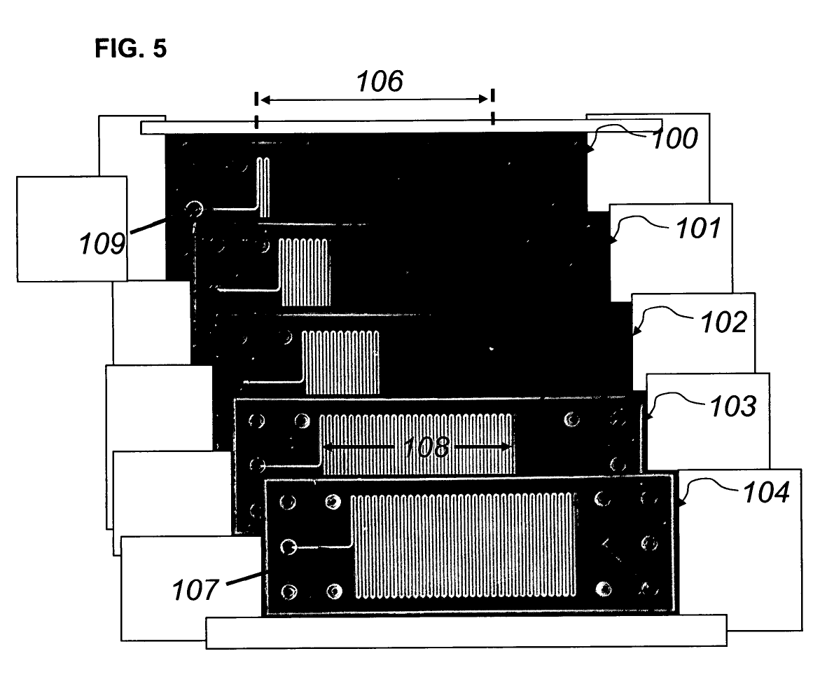

FIGS. 5 and 6 illustrate the use of the device and method of the invention in calibrating an assay strip and device. FIG. 5 is a photograph of five depletion assay chips specific for Human-IL-10 cytokine analyte, after having been run with five different concentrations of Human-IL-10 analyte, showing an increasing depletion edge length according to the IL-10 analyte concentrations run in those respective chips. This demonstrates the protein depletion assay principle using an immunoglobulin sandwich assay in one embodiment having glass microchannel chips (100, 101, 102, 103, 104) each containing a 60-cm long, curved depletion channel (107) with a channel inner dimension of about 150 micrometers and channel inlets (109) and a defined, effective flow-path total length (106).

The glass channels were oxygen-plasma activated, then homogeneously coated with a biotinylated PLL-PEG-biotin-30% copolymer (40 μl at 1 mg/ml in 10 mM Hepes Buffer pH 7.4 for 30 min). After washing with 60 μl PBS pH 7.4, the channels were incubated with streptavidin (1.66 μM; 40 μl for 10 min) and washed again (PBS pH 7.4, 60 μl). Afterwards the channels were incubated with capture agent (anti-human-IL10 antibody, 40 μl at 1 μM; overnight) and then blocked/washed with 15% fetal bovine serum (FBS) in PBS pH 7.4, 60 μl. After that, the chips were run in depletion mode by flowing 6 μl of different concentrations of human-IL-10 analyte sample through the channels at a flow rate of 0.3 μl/min (syringe pump). During that process, the analyte binds to the anti-IL-10 antibodies on the channel walls in depletion mode. After washing with PBS pH 7.4, incubation of detection antibody (anti-human-IL-10 antibody labeled with phycoerythrin, 40 μl at 100 nM in 15% FBS for 30 min), and again washing with PBS pH 7.4 (40 μl), pictures were taken of the chips in a fluorescent gel-reader apparatus equipped with a 360 nm wavelength UV black-light table and an ethidium bromide-specific filter in front of a camera. A clear depletion edge (e.g. 105) is visible on the different chips defining a specific analyte depletion length (e.g. 108), which correlates with the analyte amount (concentration) in the samples. The analyte concentrations run on the different chips were: Chip 100, 200 nM; Chip 101, 400 nM; Chip 102, 600 nM; Chip 103, 800 nM; and chip 104, 1000 nM. The designation 106 shows the total effective depletion channel length within which the sample depletion edge/length is detected.

FIG. 6 is a photograph of a depletion assay chip (110) having a sample inlet 114, demonstrating reader-less readout of the depletion length. This chip was run with biotin (PLL-PEG-biotin 30% copolymer) immobilized on the flow path walls as capture agent; streptavidin conjugated to alkaline phosphates enzyme (SA-AP) was used as the analyte. After running a specific volume and concentration of SA-AP in depletion mode through the chip, a colorimetric substrate for the AP (BCIP) was injected into the fluidic channel. In those flow path sections containing immobilized analyte on the channel walls, the enzyme transforms the transparent enzyme-substrate BCIP into a dark-colored, insoluble product. The depletion edge (111) thus becomes visible as the transition from dark to transparent in the channel, which can be seen by the unaided eye. The corresponding depletion length is shown as 112. Very high enzyme concentrations on the channel walls can lead to over-saturation of the enzyme product, making it turn transparent again, which could explain why some of the depletion length becomes transparent again (113).

Devices and methods using the principles of this invention afford simple, fast and accurate measurements in the absence of external reagents, although the use of external reagents is not outside the bounds of this invention. In some embodiments they may possess a long shelf life even at elevated temperatures, do not require external sample preparation steps, are easy to use without extensive training, and require no or at most minimal instrumentation. For these reasons, they are well suited for use in detecting and monitoring persons having diseases or conditions in resource-poor areas, including areas that experience relatively high ambient temperature, and in which highly trained personnel are scarce. However, while these features are possessed by some embodiments of this invention, the invention is not limited to such devices. For example, devices that rely on more complicated, even automated, instrumentation, are also encompassed within the scope of this invention, so long as they posses the necessary features, for example the use of analyte depletion assay and binding techniques as described herein. Such devices are useful in the determination and monitoring of large populations of subjects.

As a result of designing the devices according to the invention for simplified operator handling and instrumentation, assay complexities are transferred to the “inner parts” of the test device. Some of the most promising embodiments involve a high degree of surface-based phenomena, yet are easy to use.

The foregoing descriptions are offered primarily for purposes of illustration. Further modifications, variations and substitutions that still fall within the spirit and scope of the invention will be readily apparent to those skilled in the art. All such modifications coming within the scope of the appended claims are intended to be included therein.

All publications, patents, and patent applications cited herein are hereby incorporated by reference in their entirety for all purposes, except to the extent inconsistent with the disclosure herein.

It is claimed:

1. A method for determining the concentration, or detecting a selected concentration of an analyte in a fluid sample, comprising
(a) introducing the sample into the upstream end of an elongate, analyte-determination flow path containing along its length, an immobilized capture agent effective to bind analyte as the sample migrates through the flow path toward a downstream end of the path,
(b) allowing the sample to migrate through the flow path, wherein analyte in the sample is progressively depleted by binding to immobilized capture agent as it migrates through the path in an upstream-to-downstream direction, producing within the flow path, a region of analyte binding that terminates, at its downstream end, in a depletion end region characterized by progressively less bound analyte on progressing in an upstream-to-downstream direction along the flow path, where the distance of the depletion end region from upstream end of the path is in a specific pre-calibrated relationship to the concentration of analyte in the sample applied to the path,
(c) examining the flow path for the presence of bound analyte, thereby to determine the extent of the analyte binding region along the flow path, and
(d) from the extent of the binding region determined in step (c), determining the concentration or detecting a selected threshold concentration of the analyte in a fluid sample.

2. The method of claim 1, for use in determining the concentration of an analyte in a fluid sample, wherein step (c) includes examining the flow path for the presence of bound analyte, thereby to determine the position of the depletion end region along the path, and step (d) includes from the position of the depletion end region determined in step (c), determining the concentration of the analyte in a fluid sample.

3. The method of claim 1, for use in detecting a threshold concentration of an analyte in a fluid sample, wherein step (c) includes examining the flow path for the presence of bound analyte, thereby to determine whether the binding region in the flow path extends beyond a selected path position corresponding to a selected threshold concentration of analyte, and step (d) includes detecting a threshold concentration of analyte in the fluid sample if the binding region in the path extends beyond the selected threshold position.

4. The method of claim 1, wherein said capture agent includes binding agents, including antibodies, antibody fragments, and receptors, and nucleic acids, and the analyte is a ligand that forms a specific binding pair with the capture agent.

5. The method of claim 1, wherein said examining step includes labeling the analyte with a detectable reporter before or after steps (a) and (b), and detecting the presence of the reporter along the fluid-flow path.

6. The method of claim 1, wherein step (b) includes drawing the sample through the flow pathway by capillarity.

7. A device for determining the concentration, or detecting a selected threshold concentration of an analyte in a fluid sample, comprising:
(a) a substrate having formed therein, an elongate analyte-determination flow path containing along its length, an immobilized capture agent effective to bind analyte as a sample, when applied to the upstream end of the path, migrates through the flow path toward a downstream end of the path, wherein analyte in a sample is progressively depleted by binding to immobilized capture agent as it migrates through the flow path in an upstream-to-downstream direction, producing within the flow path, a region of analyte binding that terminates, at its downstream end, in a depletion end region characterized by progressively less bound analyte on progressing in an upstream-to-downstream direction along the flow path, where the distance along the path of the depletion end region from the upstream end of the path is in a specific pre-calibrated relationship to the concentration of analyte in the sample applied to the path, and
(b) a readout indicator disposed along a portion of the flow path for indicating
(i) analyte concentration in the sample as a function of the distance along the path of the depletion end region from the upstream end of the path, where the device is used for determining the concentration of analyte in a sample, and
(ii) a region along the path corresponding to a threshold concentration of analyte, where the device is used for detecting a threshold concentration of analyte in the sample.

8. The device of claim 7, for use in determining the concentration of an analyte in a sample, over a selected range of analyte concentrations in a sample, wherein said read-out indicator is a scale extends along a portion of the flow path corresponding to analyte concentrations within said selected range.

9. The device of claim 7, for use in detecting a threshold concentration of an analyte in a sample, wherein said read-out indicator includes a window or pointer disposed along the flow path at a position corresponding to the threshold concentration of the analyte.

10. The device of claim 7, which further includes a labeling reagent for labeling the analyte being tested with a detectable reporter.

11. The device of claim 7, wherein said substrate defines a sample-receiving reservoir in fluid communication with the fluid-flow path, and a downstream reservoir for receiving sample fluid exiting from the downstream end of the fluid-flow path.

12. The device of claim 7, wherein said flow path is a channel having a substantially fixed width along its length.

13. The device of claim 12, wherein the channel is substantially straight along its length.

14. The device of claim 12, wherein the channel has a serpentine pattern along its length.

15. The device of claim 7, for determining the concentration, or detecting a selected threshold concentration of two or more analytes, wherein the substrate has formed thereon, a plurality of such elongate fluid-flow paths, and the immobilized capture agent in each path is effective to bind one of the different analytes.

16. The device of claim 7, for determining the concentration, or detecting a selected threshold concentration of two or more analytes, wherein the fluid-flow pathway is divided into two or more analyte-specific regions, each containing immobilized capture agents effective to bind one of the different analytes.

17. The device of claim 7, wherein the flow path includes structures effective to increase the probability that the analyte, in flowing through the flow path, encounters and binds to a capture agent.

18. The device of claim 17, wherein the structures effective increase the probability that the analyte, in flowing through the flow path, encounters and binds to a capture agent, are selected from the group consisting of 3-dimensional pillars, increased surface roughness of walls forming the flow path, and beads contained in the flow path.
